SEQUENCE LISTING (12 sequences)

SEQ ID NO: 1 - DNA, 18 bp, Artificial Sequence
  Description of Artificial Sequence Synthetic primer
tgagcgcggctacagctt

SEQ ID NO: 2 - DNA, 22 bp, Artificial Sequence
  Description of Artificial Sequence Synthetic primer
tccttaatgtcacgcacgattt

SEQ ID NO: 3 - DNA, 18 bp, Artificial Sequence
  Description of Artificial Sequence Synthetic probe
accaccacggccgagcgg

SEQ ID NO: 4 - DNA, 26 bp, Artificial Sequence
  Description of Artificial Sequence Synthetic primer
ggctatcctctcagagtgacatttta

SEQ ID NO: 5 - DNA, 25 bp, Artificial Sequence
  Description of Artificial Sequence Synthetic primer
gctttatcaggttatgttgcatggt

SEQ ID NO: 6 - DNA, 15 bp, Artificial Sequence
  Description of Artificial Sequence Synthetic probe
ccactcagcagaggg

SEQ ID NO: 7 - DNA, 21 bp, Artificial Sequence
  Description of Artificial Sequence Synthetic primer
gggaatttggcgacgtaattc

SEQ ID NO: 8 - DNA, 18 bp, Artificial Sequence
  Description of Artificial Sequence Synthetic primer
gcggaggctgaggaacag

SEQ ID NO: 9 - DNA, 25 bp, Artificial Sequence
  Description of Artificial Sequence Synthetic probe
cacaggtgctctggcccagcacata

SEQ ID NO: 10 - DNA, 20 bp, Artificial Sequence
  Description of Artificial Sequence Synthetic primer
gctcccgcaaaaacttgtgt

SEQ ID NO: 11 - DNA, 19 bp, Artificial Sequence
  Description of Artificial Sequence Synthetic primer
catcgacgtccttcccaaa

SEQ ID NO: 12 - DNA, 21 bp, Artificial Sequence
  Description of Artificial Sequence Synthetic probe
accctgaggtgacacccctgc

(19) United States
(12) Patent Application Publication
(10) Pub. No.: US 2008/0293725 A1
(43) Pub. Date: Nov. 27, 2008

(54) Prognostic Molecular Markers

(76) Inventors: Rafael Rosell Costa, Barcelona (ES); Miguel Taron Roca, Barcelona (ES); Jose Maria Jimeno Donaque, Madrid (ES); Juan Carlos Tercero Lopez, Madrid (ES)

Correspondence Address: KING & SPALDING, 1185 AVENUE OF THE AMERICAS, NEW YORK, NY, 10036-4003, US

(21) Appl. No.: 11/571,589

(22) PCT Filed: Jul. 11, 2005
(86) PCT No.: PCT/EP05/07605

§ 371 Date: Feb. 5, 2007

(30) Foreign Application Priority Data
Jul. 9, 2004 (EP) 04076997.8

(51) Int. Cl. A61K 31/495; C12Q 1/68; A61P 31/00

(52) U.S. Cl. 514/249; 435/6

(57) ABSTRACT

The present invention relates to the use of ecteinascidin 743 in patients having certain levels of molecular markers who can predict the outcome of chemotherapy, in particular in patients having low levels of BRCA1 expression.

### FIELD OF THE INVENTION

The present invention relates to the use of ecteinascidin 743, and more specially to the use of ecteinascidin 743 in patients having certain levels of molecular markers, in particular having low levels of BRCA1 expression.

### BACKGROUND OF THE INVENTION

Cancer comprises a group of malignant neoplasms that can be divided into two categories: carcinoma, comprising a majority of the cases observed in the clinics, and other less frequent cancers, which include leukemia, lymphoma, central nervous system tumors and sarcoma. Carcinomas have their origin in epithelial tissues while sarcomas develop from connective tissues and those structures that had their origin in mesoderm tissues. Sarcomas can affect, for instance, muscle or bone and occur in the bones, bladder, kidneys, liver, lung, parotid, spleen, etc.

Cancer is invasive and tends to metastasise to new sites. It spreads directly into surrounding tissues and also may be disseminated through the lymphatic and circulatory systems.

Many treatments are available for cancer, including surgery and radiation, for localised disease, and drugs. However, the efficacy of available treatments on many cancer types is limited and new improved forms of treatment showing clinical benefit are needed.

This is especially true for those patients that present the disease in advanced and/or metastatic state. It is also true for patients relapsing with progressive disease after having been previously treated with established therapies for which further treatment with the same therapy is mostly ineffective due to the acquisition of resistance or to limitations in the administration of the therapies because of associated toxicities.

Chemotherapy plays a significant part in cancer treatment, as it is required for treatment of advanced cancers with distant metastasis and often helpful for tumor reduction before surgery. Many anti-cancer drugs have been developed based on various modes of action.

The most commonly used types of anticancer agents include: DNA-alkylating agents (for example, cyclophosphamide, ifosfamide), antimetabolites (for example, methotrexate, a folate antagonist, and 5-fluorouracil, a pyrimidine antagonist), microtubule disrupters (for example, vincristine, vinblastine, paclitaxel), DNA intercalators (for example, doxorubicin, daunomycin, cisplatin), and hormone therapy (for example, tamoxifen, flutamide). The ideal antineoplastic drug would kill cancer cells selectively, with a wide therapeutic index relative to its toxicity towards non-malignant cells. It would also retain its efficacy against malignant cells, even after prolonged exposure to the drug. Unfortunately, none of the current chemotherapies possess an ideal profile. Most possess very narrow therapeutic indexes and, in practically every instance, cancerous cells exposed to slightly sublethal concentrations of a chemotherapeutic agent will develop resistance to such an agent, and quite often cross-resistance to several other antineoplastic agents.

The ecteinascidins (herein abbreviated ETs) are exceedingly potent antitumor agents isolated from the marine tunicate Ecteinascidia turbinata. Several ecteinascidins have been reported previously in the patent and scientific literature. See, for example U.S. Pat. No. 5,089,273, which describes novel compounds of matter extracted from the tropical marine invertebrate, Ecteinascidia turbinata, and designated therein as ecteinascidins 729, 743, 745, 759A, 759B and 770. These compounds are useful as antibacterial and/or antitumor agents in mammals. U.S. Pat. No. 5,478,932 describes other novel ecteinascidins isolated from the Caribbean tunicate Ecteinascidia turbinata, which provide in vivo protection against P388 lymphoma, B16 melanoma, M5076 ovarian sarcoma, Lewis lung carcinoma, and the LX-1 human lung and MX-1 human mammary carcinoma xenografts.

One of the ETs, ecteinascidin-743 (ET-743), is a tetrahydroisoquinoline alkaloid with considerable in vitro and in vivo antitumor activity in murine and human tumors, and potent antineoplastic activity against a variety of human tumor xenografts grown in athymic mice, including melanoma and ovarian and breast carcinoma.

This compound is presently in clinical trials. A clinical development program of ET-743 in cancer patients was started with phase I studies investigating 1-hour, 3-hour, 24-hour and 72-hour intravenous infusion schedules and a 1 hour daily×5 (d×5) schedule. Promising responses were observed in patients with sarcoma and breast and ovarian carcinoma. Therefore this new drug is currently under intense investigation in several phase II clinical trials in cancer patients with a variety of neoplastic diseases. Further detail on the use of ET-743 for the treatment of cancer in the human body is given in WO 00 69441, WO 02 36135 and WO 0339571, incorporated herein by reference in their entirety.

A recent review of ET-743, its chemistry, mechanism of action and preclinical and clinical development can be found in Kesteren, Ch. Van et al., 2003, Anti-Cancer Drugs, 14 (7), pages 487-502: “ET-743 (trabectedin, ET-743): the development of an anticancer agent of marine origin”, and references therein.

During the past 30 years medical oncologists have focused to optimise the outcome of cancer patients and it is just now that the new technologies available are allowing to investigate polymorphisms, gene expression levels and gene mutations aimed to predict the impact of a given therapy in different groups of cancer patients to tailor chemotherapy. Representative examples include the relation between the TS mRNA expression and the response and the survival with antifolates, beta tubulin III mRNA levels and response to tubulin interacting agents, PTEN methylation and resistance to CPT-11 and STAT3 over expression and resistance to EGF interacting agents.

A molecular observation of potential clinical impact relates to the paradoxical relation between the efficiency of the NER pathway and the cytotoxicity of ET-743. In fact, tumour cells that are efficient in this DNA repair pathway appear to be more sensitive to ET-743. This evidence is in contrast with the pattern noted with platin based interventions that are highly dependent to the activity of this repair pathway.

There is a strong evidence on the key role of NER pathways on the cytotoxicity of ET-743 in cell lines. ET-743 binds to G residues in the minor groove of DNA forming adducts that distorted the DNA helix structure and they are recognised by NER mechanisms. Takebayasi et al. (Nature Medicine, 7(8), 961-966, August 2001) have proposed that the presence of these DNA adducts in transcribed genes, blocks the Transcription Coupled NER (TC-NER) system by stalling the cleavage intermediates and producing lethal Single Strand Breaks (SSBs).

Breast Cancer 1 (BRCA1) plays a crucial role in DNA repair, and decreased BRCA1 mRNA expression has been observed in both sporadic and hereditary breast cancers (Kennedy R D. et al. Lancet, 2002, 360, 1007-1014). BRCA1 is implicated in transcription-coupled nucleotide excision repair (TC-NER), and modulation of its expression leads to modification of TC-NER and hence to radio- and chemoresistance.

Upregulation of BRCA1 expression led to increased cisplatin resistance in the SKOV-3 human ovarian cancer cell line (Husain A. et al. Cancer Res. 1998, 58, 1120-1123), and restoration of BRCA1 in the BRCA1-negative HCC1937 human breast cancer cell line restored radioresistance (Abbott D W. et al. J Biol Chem. 1999, 274, 18808-18812).

BRCA1 is also involved in homologous recombination repair (HRR) and non-homologous end joining in response to DNA damage (Mullan P B. et al. Oncogene, 2001, 20, 6123-6131). In addition, it is a component of a large DNA repair complex termed the BRCA1-associated genome surveillance complex, which contains a number of mismatch repair proteins, indicating a potential role for BRCA1 in mismatch repair (Kennedy R D. et al. Lancet, 2002, 360, 1007-1014).

BRCA1 may also be a regulator of mitotic spindle assembly, as BRCA1 and β-tubulin colocalize to the microtubules of the mitotic spindle and to the centrosomes (Lotti L V. et al. Genes, Chromosomes & Cancer, 2002, 35, 193-203).

Enhanced BRCA1 expression has been linked to apoptosis through the c-Jun N-terminal kinase pathway (Harkin D P. et al. Cell, 1999, 97, 575-586), which is activated by cisplatin-induced DNA damage; inhibition of this pathway increased cisplatin sensitivity in cell lines (Potapova O. et al. J Biol Chem. 1997, 272, 14041-14044).

Decreased BRCA1 mRNA expression in a breast cancer cell line, as determined by real-time quantitative polymerase chain reaction (RT-QPCR), led to greater sensitivity to cisplatin and etoposide but to greater resistance to the microtubule-interfering agents paclitaxel and vincristine (Lafarge S. et al. Oncogene, 2001, 20, 6597-6606).

Reconstitution of wild-type BRCA1 into the BRCA1-negative HCC1937 breast cancer cell line (Tomlinson G E. et al. Cancer Res. 1998, 58, 3237-3242) resulted in a 20-fold increase in cisplatin resistance and, in contrast, in a 1000-10.000-fold increase in sensitivity to antimicrotubule drugs (paclitaxel and vinorelbine) (Mullan P B. et al. Oncogene, 2001, 20, 6123-6131, and Kennedy R D. et al. Proc Am Soc Clin Oncol. 2003, 22, 848). Mouse models carrying conditional disruption of BRCA1 were highly sensitive to doxorubicin and gamma irradiation but resistant to tamoxifen, providing additional evidence for differential chemosensitivity linked to BRCA1 expression (Brodie S G. et al. Oncogene, 2001, 20, 7514-7523).

When BRCA1 expression was examined by semi-quantitative PCR in women with sporadic breast cancer, lower BRCA1 mRNA levels (bottom quartile) were associated with a higher frequency of distant metastases (Seery L T. et al. Int J Cancer (Pred Oncol), 1999, 84, 258-262).

Despite the wealth of data in cell lines and mouse models, only one small study has examined the correlation of BRCA1 and BRCA2 mRNA expression with response to chemotherapy in the clinical setting. Among 25 women with docetaxel-treated locally advanced or metastatic breast cancer (Egawa C. et al. Int J Cancer (Pred Oncol), 2001, 95, 255-259), both BRCA1 and BRCA2 mRNA levels were lower in responders than in non-responders, though the difference was statistically significant only for BRCA2.

### SUMMARY OF THE INVENTION

It is an object of the invention to provide an efficacious use of ET-743 for the treatment of cancer. More particularly, an object of this invention is to provide an effective use of ecteinascidin 743 in patients having certain levels of molecular markers, and in particular having low levels of BRCA1 expression.

Therefore, according to the present invention, we provide an efficacious use of ET-743 for the treatment of cancer in patients having low levels of BRCA1 expression.

We also provide the use of BRCA1 as a marker for the selection of cancer patients to be efficaciously treated with ET-743.

In another aspect the invention is directed to a method of treating cancer in a patient, the method comprising the steps of: assaying a biological sample from the individual for BRCA1 expression level, and when the expression level is low, treating the patient with ET-743.

### BRIEF DESCRIPTION OF THE FIGURES

FIG. 1. Kaplan and Meier plots of the patients included in the study.

FIG. 2A. Kaplan and Meier plots of PFS and Survival of patients according to its BRCA1 mRNA expression levels.

FIG. 2B. Kaplan and Meier plots of PFS and Survival of patients according to its ERCC1 mRNA expression levels.

FIG. 2C. Kaplan and Meier plots of PFS and Survival of patients according to its XPD mRNA expression levels.

### DETAILED DESCRIPTION

ET-743 is a natural compound represented by the following formula:

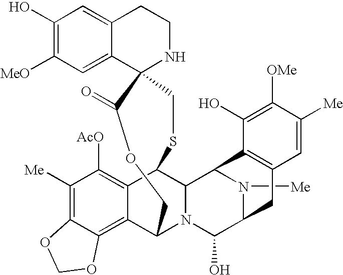

As used herein, the term “ET-743” also covers any pharmaceutically acceptable salt, ester, solvate, hydrate or a prodrug compound which, upon administration to the patient is capable of providing (directly or indirectly) the compound ET-743. The preparation of salts and other derivatives, and prodrugs, can be carried out by methods known in the art.

As single agent ET-743 has proven to induce long lasting objective remissions and tumor control in subsets of patients harbouring sarcomas relapsed to conventional therapy, ovarian cancer resistant or relapsed to Cisplatin-Paclitaxel and in breast cancer patients exposed to doxorubicin and to taxanes.

Now, we have found that BRCA1 mRNA expression can also play an important role in predicting differential chemotherapy sensitivity in cancer patients treated with ET-743.

Thus in one aspect the invention is directed to the use of ET-743 in the manufacture of a medicament for the treatment of cancer patients having low levels of BRCA1 gene expression.

The values for “low,” “normal,” or “high” levels of expression are determined by comparison to reproducible standards which correspond to the median value of expression levels of BRCA1 measured in a collection of tumor tissue in biopsy samples from cancer patients, previous to the ET-743 treatment. Once this median value is established, the level of this marker expressed in tumor tissues from patients can be compared with this median value, and thus be assigned a level of “low,” “normal” or “high.”

The measure of relative gene expression is preferably made by using β-actin as an endogenous control, although other methods known in the art can be used, as long as relative levels of BRCA1 can be assigned to the samples. Levels of mRNA or the corresponding protein can be measured to obtain the relative level of BRCA1 expression. Standard methods of measurement well known in the art are used.

The collection of samples from which the reference level is derived will preferably be constituted from patient suffering from the same type of cancer. For example, the one described in the examples which is statistically representative was constituted with 61 samples from sarcoma patients. In any case it can contain a different number of samples.

In a particular embodiment, the expression level is determined using RNA obtained from a formalin-fixed, paraffin-embedded tissue sample. Other tissue samples are envisaged, such as fresh tissue from a biopsy or blood samples depending on their availability.

While all techniques of gene expression profiling, as well as proteomics techniques, are suitable for use in performing the foregoing aspects of the invention, the gene expression levels are often determined by reverse transcription polymerase chain reaction (RT-PCR).

We have evaluated if expression levels of the DNA repair genes XPD, ERCC1 and BRCA1 may induce differential sensitivity to ET-743 in cancer patients, for example in sarcoma patients. We have found that the marker gene having a greater correlation to the clinical outcome is BRCA1. Surprisingly, subdivision of the full cohort of patients in two equal subpopulations (“low” level of expression and “high” level of expression) according to the BRCA1 expression produces a significant increase of the efficiency of ET-743 in the target subpopulation from 16% to 21% for objective response (partial response+minor response (PR+MR)) and 24% to 38% for progression free survival higher than 6 months (PFS6).

On the other hand, we have found that ERCC1 and XPD expression levels do not impact the clinical outcome of the ET-743 therapy, indicating that ET-743 would be equally active in those patients with poor response to Cisplatin or Doxorubicin due to the high expression levels of ERCC1 and XPD.

Accordingly, the present invention relates to the use of ET-743 for the treatment of cancer in patients having low levels of BRCA1. Treatment of cancer patients with a BRCA1 level <3 is preferred, and a BRCA1 level lower than 2 is the most preferred.

In one embodiment relative gene expression quantification is calculated according to the comparative Ct method using β-actin as an endogenous control and commercial RNA controls as calibrators. Final results, are determined according to the formula 2⁻⁽^{^{Δ}}^{Ct sample-}^{^{Δ}}^{Ct calibrator)}, where ΔCT values of the calibrator and sample are determined by subtracting the CT value of the target gene from the value of the β-actin gene.

ET-743 is typically supplied and stored as a sterile lyophilized product which comprises ET-743 and pharmaceutically acceptable excipients in a formulation adequate for therapeutic use, in particular a formulation containing mannitol and a phosphate salt buffered to an adequate pH.

It is currently preferred to administer the ET-743 by infusion. The infusing step is typically repeated on a cyclic basis, which may be repeated as appropriate over for instance 1 to 20 cycles. The cycle includes a phase of infusing ET-743, and usually also a phase of not infusing ET-743. Typically the cycle is worked out in weeks, and thus the cycle normally comprises one or more weeks of an ET-743 infusion phase, and one or more weeks to complete the cycle. A cycle of 3 weeks is preferred, but alternatively it can be from 2 to 6 weeks. The infusion phase can itself be a single administration in each cycle of say 1 to 72 hours, more usually of about 1, 3 or 24 hours; or an infusion on a daily basis in the infusion phase of the cycle for preferably 1 to 5 hours, especially 1 or 3 hours; or an infusion on a weekly basis in the infusion phase of the cycle for preferably 1 to 3 hours, especially 2 or 3 hours. We currently prefer a single administration at the start of each cycle. Preferably the infusion time is about 1, 3 or 24 hour.

The dose will be selected according to the dosing schedule, having regard to the existing data on Dose Limiting Toxicity, on which see for example the above mentioned WO 00 69441 WO 02 36135 and WO 03 39571 patent specifications, and also see Kesteren, Ch. Van et al., 2003, Anti-Cancer Drugs, 14 (7), 487-502. This article is also incorporated herein in full by specific reference.

Representative schedules and dosages are for example:

a) about 1.5 mg/m2 body surface area, administered as an intravenous infusion over 24 hours with a three week interval between cycles;

b) about 1.3 mg/m2 body surface area, administered as an intravenous infusion over 3 hours with a three week interval between cycles;

c) about 0.580 mg/m2 body surface area, administered weekly as an intravenous infusion over 3 hours during 3 weeks and one week rest.

As noted in the article by Kesteren et al. (2003), the combination of ET-743 with dexamethasone gives unexpected advantages. It has a role in hepatic prophylaxis. We therefore prefer to administer dexamethasone to the patient, typically at around the time of infusing the ET-743. For example, we prefer to give dexamethasone on the day before ET-743, and/or the day after ET-743. The administration of dexamethasone can be extended, for example to more than one day following ET-743. In particular, we prefer to give dexamethasone at days—1, 2, 3 and 4 relative to a single administration of ET-743 on day 1 of a cycle.

In the use according to the present invention the compound ET-743 may be used with other drugs to provide a combination therapy. The other drugs may form part of the same composition, or be provided as a separate composition for administration at the same time or a different time. The identity of the other drug is not particularly limited, and suitable candidates include: a) drugs with antimitotic effects, especially those which targetcytoskeletal elements, including microtubule modulators such as taxane drugs (such as taxol, paclitaxel, taxotere, docetaxel), podophylotoxins or vinca alkaloids (vincristine, vinblastine); b) antimetabolite drugs (such as 5-fluorouracil, cytarabine, gemcitabine, purine analogues such as pentostatin, methotrexate); c) alkylating agents or nitrogen mustards (such as nitrosoureas, cyclophosphamide or ifosphamide); d) drugs which target DNA such as the antracycline drugs adriamycin, doxorubicin, pharmorubicin or epirubicin; e) drugs which target topoisomerases such as etoposide; hormones and hormone agonists or antagonists such as estrogens, antiestrogens (tamoxifen and related compounds) and androgens, flutamide, leuprorelin, goserelin, cyprotrone or octreotide; g) drugs which target signal transduction in tumour cells including antibody derivatives such as herceptin; h) alkylating drugs such as platinum drugs (cis-platin, carbonplatin, oxaliplatin, paraplatin) or nitrosoureas; i) drugs potentially affecting metastasis of tumours such as matrix metalloproteinase inhibitors; j) gene therapy and antisense agents; k) antibody therapeutics; l) other bioactive compounds of marine origin, notably the didemnins such as aplidine; m) steroid analogues, in particular dexamethasone; n) anti-inflammatory drugs, including nonsteroidal agents (such as acetaminophen or ibuprofen) or steroids and their derivatives in particular dexamethasone; and o) anti-emetic drugs, including 5HT-3 inhibitors (such as palonisetron, gramisetron or ondasetron).

Depending on the type of tumor and the developmental stage of the disease, the treatments of the invention are useful in preventing the risk of developing tumors, in promoting tumor regression, in stopping tumor growth and/or in preventing metastasis. In particular, the method of the invention is suited for human patients, especially those who are relapsing or refractory to previous chemotherapy. First line therapy is also envisaged.

Although guidance for the dosage is given above, the correct dosage of the compound will vary according to the particular formulation, the mode of application, and the particular situs, host and tumor being treated. Other factors like age, body weight, sex, diet, time of administration, rate of excretion, condition of the host, drug combinations, reaction sensitivities and severity of the disease shall be taken into account. Administration can be carried out continuously or periodically within the maximum tolerated dose.

The use of ET-743 according to the invention is particularly preferred for the treatment of sarcoma, leiomyosarcoma, liposarcoma, osteosarcoma, ovarian cancer, breast cancer, melanoma, colorectal cancer, mesothelioma, renal cancer, endometrial cancer and lung cancer; preferably sarcomas, most preferably leiomyosarcoma, liposarcoma or osteosarcoma.

### EXAMPLE 1

### Sample and Clinical Data Collection

In this study, 61 paraffin embedded tumoral samples from sarcoma patients before the treatment with any chemotherapy agent have been evaluated.

The majority of patients were treated before with one or several chemotherapy agents and later they followed a treatment with ET-743. The dosage of intravenous infusion ET-743 given to the different patients was within the range of 1.650-1.0 mg/m2; the schedules were 24 hours or 3 hour IV infusion with a three week interval between cycles; and the number of cycles ranged from 1 up to 25 cycles in some patients.

The clinical data from the patients was collected in the clinical data collection form and matched with the molecular data after completion of the mRNA expression levels determination (Table 1).

Quantification of mRNA Expression Levels

We examined XPD, ERCC1 and/or BRCA1 gene expression in formalin-fixed, paraffin-embedded tumor specimens from the 61 patients as previously described (Specht K. et al. Am J Pathol, 2001, 158, 419-429 and Krafft A E. et al. Mol Diagn. 1997, 3, 217-230). After standard tissue sample deparaffinisation using xylene and alcohols, samples were lysed in a tris-chloride, EDTA, sodium dodecyl sulphate (SDS) and proteinase K containing buffer. RNA was then extracted with phenol-chloroform-isoamyl alcohol followed by precipitation with isopropanol in the presence of glycogen and sodium acetate. RNA was resuspended in RNA storage solution (Ambion Inc; Austin Tex., USA) and treated with DNAse I to avoid DNA contamination. cDNA was synthesized using M-MLV retrotranscriptase enzyme. Template cDNA was added to Taqman Universal Master Mix (AB; Applied Biosystems, Foster City, Calif., USA) in a 20-μl reaction with specific primers and probe for each gene. The primer and probe sets were designed using Primer Express 2.0 Software (AB). Quantification of gene expression was performed using the ABI Prism 7900HT Sequence Detection System (AB). The primers and 5′ labeled fluorescent reporter dye (6FAM) probe were as follows: β-actin: forward 5′ TGA GCG CGG CTA CAG CTT 3′, reverse 5′ TCC TTA ATG TCA CGC ACG ATT T 3′, probe 5′ ACC ACC ACG GCC GAG CGG 3′; BRCA1: forward 5′ GGC TAT CCT CTC AGA GTG ACA TTT TA 3′, reverse 5′ GCT TTA TCA GGT TAT GTT GCA TGG T 3′, probe 5′ CCA CTC AGC AGA GGG 3′; ERCC1: forward 5′ GGG AAT TTG GCG ACG TAA TTC 3′, reverse 5′ GCG GAG GCT GAG GAA CAG 3′, probe 5′CAC AGG TGC TCT GGC CCA GCA CAT A 3′; XPD: forward 5′ GCT CCC GCA AAA ACT TGT GT 3′, reverse 5′ CAT CGA CGT CCT TCC CAA A 3′, probe 5′ ACC CTG AGG TGA CAC CCC TGC 3′.

Relative gene expression quantification was calculated according to the comparative Ct method using β-actin as an endogenous control and commercial RNA controls (Stratagene, La Jolla, Calif.) as calibrators. Final results, were determined as follows: 2⁻⁽^{^{Δ}}^{Ct sample-}^{^{Δ}}^{Ct calibrator)}, where ΔCT values of the calibrator and sample are determined by subtracting the CT value of the target gene from the value of the β-actin gene. In all experiments, only triplicates with a standard deviation (SD) of the Ct value <0.20 were accepted. In addition, for each sample analyzed, a retrotranscriptase minus control was run in the same plate to assure lack of genomic DNA contamination.

### Statistical Methods

SAS v8.2 (statistical software) was used for all the statistical analysis. The statistical techniques for univariate, bivariate and multivariate variables were chosen, according with the nature of variables that will be analysed, i.e. when the dependent variable is a temporal variable with censor status the Cox regression would be applied, when correlation between variables will be computed the Pearson and/or Spearman measures would be used. P-values below 0.05 will be considered statistically significant in all tests, when appropriate 95% confidence intervals will be presented too.

### Results

A total of 61 paraffin embedded tumor samples were evaluated. Table 1 shows the most relevant clinical and molecular data of the 61 patients (CR: Complete Response; PR: Partial Response; MR: Minor Response; SD: Stable Disease; PD: Progressive Disease; OS: Overall survival; PFS: progression-free survival). These samples came from sarcoma patients before being treated with a chemotherapy agent.

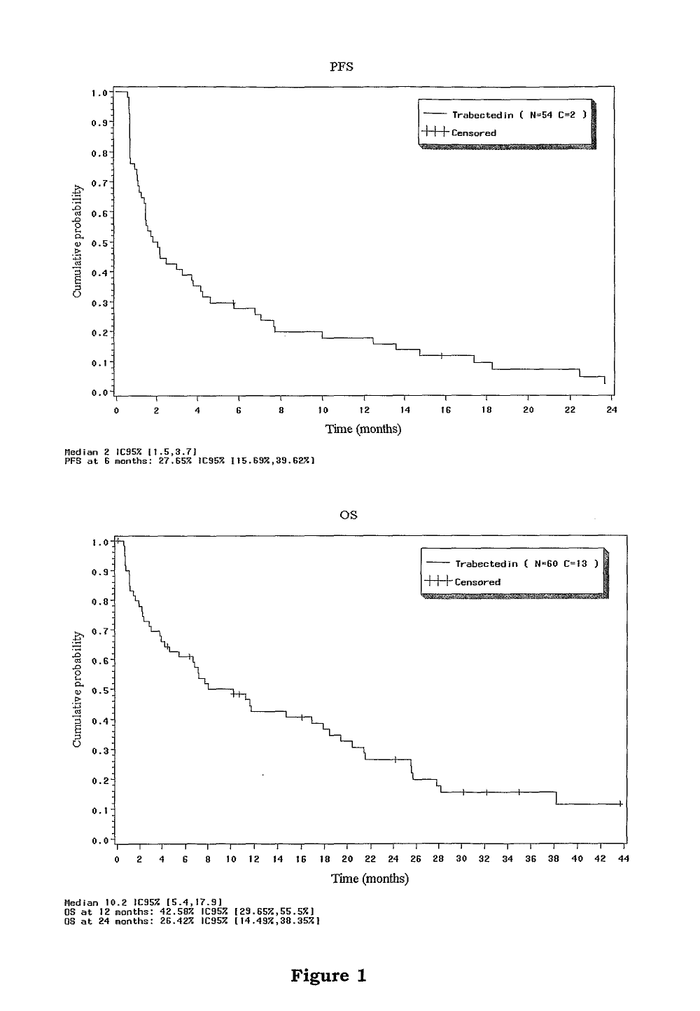

After treatment with ET-743, the overall response rate (RR) in 55 evaluable patients was 15% when considering only Partial Responses (8 PR/55 evaluable patients) or 16% when Minor Responses (MR) were also considered (8 PR+1 MR/55 patients). Also, 15 patients (27%) when Stable Disease (SD) were also considered ((8 PR+1 MR+6 SD)/55 patients) achieved progression free survival ≧6 months (PFS6). The median duration of the response (PR+MR) was 13.6 months (range 44.1 to 3.8 months) and 6 out of 15 SD reached the PFS6. Median survival was 7.7 months (0.1-66.9 months), although 14 patients are still censored. The overall progression free survival at 6 months (Kaplan-Meier) is 27.65% and the median survival is 10.2 months (FIG. 1).

TABLE 1
Clinical and molecular data of the 61 patients

| | Clinical Parameters | | | | | | | |
|---|---|---|---|---|---|---|---|---|
| | | | | PFS | OS | mRNA expression | | |
| Patient # | Tumor Histology | # Cycles | Response | months | months | ERCC1 | BRCA-1 | XPD |
| 1 | Leiomyosarcoma | 1 | PD | 1.5 | 6.7 | | 0.35 | |
| 2 | Liposarcoma | 2 | PD | 1.7 | 2.4 | | 0.38 | |
| 3 | Synovial Sarcoma | 11 | SD | 7.7 | 11.7 | | 0.45 | |
| 4 | Extra esqueletal osteosarcoma | 1 | PD | 0.7 | 2.9 | 5.53 | 0.54 | 2.14 |
| 5 | Osteosarcoma | 6 | SD | 4.2 | 11.7 | 4.42 | 0.58 | 1.42 |
| 6 | Leiomyosarcoma | 25 | SD | 23.7 | 16.1 | 1.01 | 0.58 | 0.9 |
| 7 | Léiomyosarcome | | PR | 17.4 | 43.7 | | 0.59 | |
| 8 | Sarcoma NOS | 10 | SD | 7.7 | 25.7 | | 0.67 | |
| 9 | Ductal Carcinoma | 1 | NE | | 1.9 | 5.82 | 0.77 | 1.99 |
| 10 | MPNST | 2 | PD | 2.1 | | | 0.80 | |
| 11 | Extra esqueletal osteosarcoma | 2 | PD | 1.5 | 11.3 | 3.49 | 0.85 | 0.94 |
| 12 | Leiomyosarcoma | 8 | PR | 44.1 | 64.3 | 4.76 | 0.98 | 0.9 |
| 13 | Leiomyosarcoma | 2 | NE | | 1.6 | 1.87 | 0.99 | 0.58 |
| 14 | Synovial sarcoma | 2 | PD | 0.7 | 7.2 | 5.7 | 1.03 | 2.27 |
| 15 | Ostéosarcoma | 4 | SD | 2.5 | 16.9 | 5.82 | 1.03 | 1.49 |
| 16 | Leiomyosarcoma (GIST) | 6 | SD | 5.7 | 66.9 | 5.12 | 1.23 | 2.66 |
| 17 | Osteosarcoma | 2 | PD | 0.7 | 0.7 | 5.59 | 1.24 | 0.99 |
| 18 | Synovial Sarcoma | 2 | PD | 1.1 | 3.9 | 10.65 | 1.27 | 3.19 |
| 19 | Alveolar Softcell Sarcoma | 2 | PD | 0.7 | 2.2 | 2.22 | 1.31 | 0.8 |
| 20 | Carcinosarcoma | 3 | PD | 2.2 | 7.7 | 10.68 | 1.36 | 3.19 |
| 21 | Osteosarcoma | 2 | PD | 0.9 | 35.0 | 4.31 | 1.38 | 1.75 |
| 22 | Leiomyosarcoma | 11 | PR | 10.0 | 30.2 | 2.34 | 1.45 | 1.28 |
| 23 | Leiomyosarcoma | 10 | PR | 13.6 | 20.4 | 7.46 | 1.49 | 2.67 |
| 24 | Spindel cell sarcoma unclassified | 16 | SD | 15.8 | 18.4 | 9.45 | 1.81 | 2.03 |
| 25 | Ostéosarcome osseux | | PD | 1.5 | 6.4 | | 1.93 | |
| 26 | Synovial sarcoma monophasic | 1 | MR | 7.1 | 10.2 | 11.23 | 1.97 | 3.07 |
| 27 | Sarcome d'Ewing | | | | | | 2.00 | |
| 28 | PNET | 1 | PD | 0.7 | 0.8 | 7.03 | 2.02 | 4.04 |
| 29 | Sarcome Stromal Utérin | | MR | 3.8 | | | 2.11 | |
| 30 | Leiomyosarcoma | 2 | PD | 0.7 | 3.0 | 3.24 | 2.19 | 1.54 |
| 31 | MFH | 2 | PD | 1.6 | 4.5 | 2.03 | 2.19 | 1.05 |
| 32 | Leiomyosarcoma | 6 | SD | 4.6 | 21.5 | | 2.19 | |
| 33 | Liposarcoma | 2 | NE | | 1.1 | 5.9 | 2.35 | 2.44 |
| 34 | myxoid liposarcoma | 20 | PR | 22.4 | 28.2 | | 2.38 | |
| 35 | ORCT | 2 | PD | 1.4 | 5.4 | | 2.44 | |
| 36 | Sarcoma NOS | 4 | SD | 3.7 | 14.7 | | 2.55 | |
| 37 | Leiomyosarcoma | 2 | PD | 1.3 | 21.4 | | 2.83 | |
| 38 | Synovial sarcoma | 4 | SD | 3.2 | 19.4 | 22.46 | 2.92 | 5.06 |
| 39 | Leiomyosarcoma osteogénico | 2 | PD | 0.7 | 1.1 | 6.27 | 2.99 | 1.5 |
| | extraóseo | | | | | | | |
| 40 | Liposarcoma | 2 | PD | 0.7 | 3.8 | 3.9 | 3.46 | 1.65 |
| 41 | Synovial sarcoma | 3 | PD | 2.2 | 4.2 | 16.49 | 3.59 | 4.42 |
| 42 | Leiomyosarcoma | 1 | PD | 0.6 | 0.6 | 5.8 | 3.96 | 1.88 |
| 43 | MFH; called high grade | 5 | SD | 4.3 | 24.2 | 5.91 | 4.69 | 3.71 |
| | liposarcoma in 1990 | | | | | | | |
| 44 | Low grade Leiomyosarcoma | 2 | PD | 1.5 | 1.5 | 3.75 | 4.74 | 2.83 |
| 45 | Osteosarcoma | 8 | PR | 18.3 | 25.5 | 11.9 | 4.8 | 3.02 |
| 46 | Ewing Sarcoma | 1 | PD | 1.0 | | | 4.92 | |
| 47 | Pleiomorf sarcoma (Histiocytoma) | 1 | NE | | 0.7 | 7.72 | 4.96 | 1.5 |
| 48 | Synovial Sarcoma | 7 | SD | 6.8 | 17.9 | | 5.54 | |
| 49 | Leiomyosarcoma | 1 | PD | 0.7 | 1.1 | 19.99 | 5.88 | 13.38 |
| 50 | Malignat peripheral nerve sheet | 1 | PD | 0.7 | 4.7 | 6.71 | 6.53 | 1.16 |
| | tumor | | | | | | | |
| 51 | Dedif. Liposarcoma | 1 | PD | 1.1 | 10.2 | | 7.08 | |
| 52 | Fibrous tumor | 2 | PD | 0.7 | 0.7 | 19.43 | 7.62 | 12.46 |
| 53 | Osteosarcoma | 3 | PD | 1.8 | 7.1 | 22.94 | 7.67 | 8.68 |
| 54 | Ewing sarcoma (PNET) | 2 | PD | 0.7 | 0.7 | 16.51 | 8.44 | 5.31 |
| 55 | Sarcome d'Ewing | | PD | | 0.1 | | 11.04 | |
| 56 | Synovial sarcoma | 6 | SD | 5.8 | 6.8 | 22.94 | 11.14 | 11.37 |
| 57 | Neurogenic sarcoma | 1 | NE | | 38.2 | 17.18 | | 3.72 |
| 58 | Osteosarcoma | 4 | SD | 3.0 | 25.6 | 2.38 | | 1.97 |
| 59 | Mixoid/round cell liposarcoma | 14 | PR | 12.4 | 27.8 | 6.39 | | |
| 60 | Synovial sarcoma monophasic | 13 | SD | 14.7 | 32.2 | 6.9 | | 2.13 |
| 61 | Leiomyosarcoma | 2 | PD | 1.2 | 8.1 | 2.35 | | 0.91 |

1. Correlation of BRCA1 mRNA Expression Levels and ET-743 Treatment Outcome.

The association between the expression levels of BRCA1 mRNA with the clinical outcome of the patients is shown in Table 2A.

TABLE 2A
Association of mRNA expression of BRCA1
and patients clinical outcome.

BRCA1 mRNA

| | <1.97 | >=1.97 | Total |
|---|---|---|---|
| PFS | | | |
| <6 | 16 | 22 | 38 |
| >=6 | 9 | 3 | 12 |
| | 36% | 12% | |
| Total | 25 | 25 | 34 |
| | 50% | 50% | |

Frequency Missing = 11

| | | | |
|---|---|---|---|
| resp | | | |
| PR + MR | 5 | 3 | 8 |
| | 21% | 11% | |
| SD | 7 | 6 | 13 |
| PD | 12 | 18 | 30 |
| Total | 24 | 27 | 51 |
| | 51% | 49% | |

Frequency Missing = 10

The amount of BRCA1 mRNA relative to the β-actin (internal control) was determined in 56 samples ranging from 0.35 to 11.14, a 32-fold difference from the minimum to the maximum value found. The median expression value was 1.97.

Table 2A shows that patients reaching the PFS6 endpoint 9 out of 12 (75%) had BRCA1 expression values under the median value (1.97) of the cohort. Similarly, 5 of 8 (63%) patients having objective response (PR+MR) have expression values of BRCA1 under the median value.

The probability of reaching PFS6 or having objective response is statistically significant higher in those patients having BRCA1 expression lower than the median value. In fact, 9 out of 24 (38%) of patients expression low BRCA1 reach PFS6 vs 3 of 26 (12%) of the high expression of BRCA1. Similarly, 5 out of 24 (21%) low expressers of BRCA1 had objective response (PR+MR) compared to 3 in 27 (11%) of high expression patients. The fact that the correlation is significant with clinical response and FPFS6 indicate that BRCA1 expression level is a marker of the treatment with ET-743 and not a marker of tumor aggressiveness.

This means that, subdivision of the full cohort of patients in two equal subpopulations according to the BRCA1 expression produces an increase of the efficiency of ET-743 in the target subpopulation from 16% (8/51) to 21% (5/24) in objective response (1.3 fold increase) and from 24% (12/50) to 38% (9/24) in PFS6 rates (1.6 fold increase).

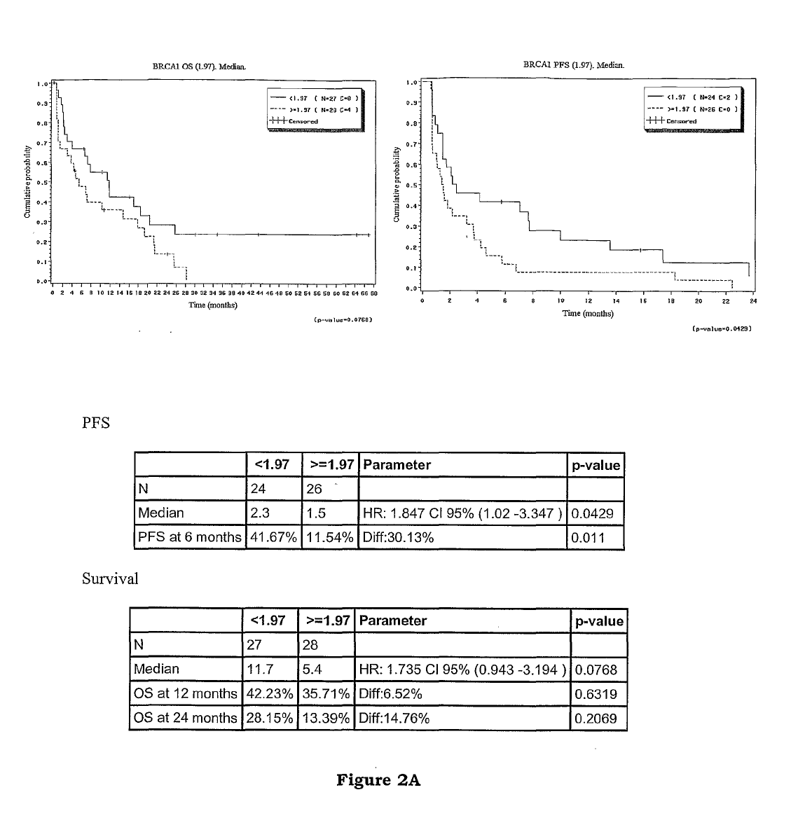

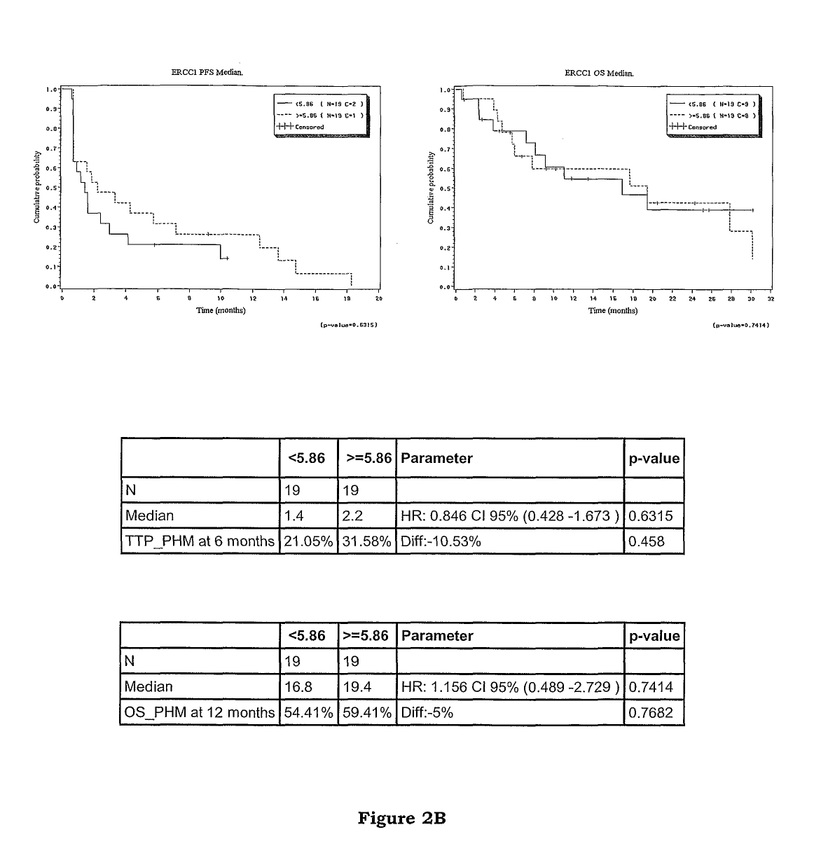

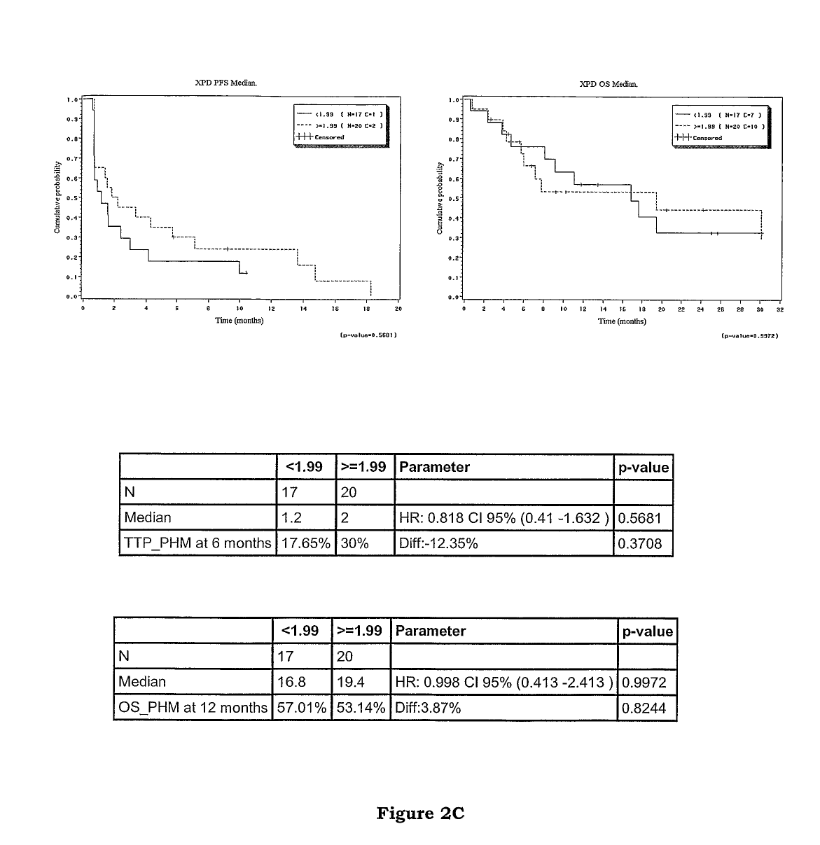

The Kaplan-Meier plots of FIG. 2A show a statistically significant difference [p=0.043] in PFS and a clear difference [p=0.077] in survival on those patients having a BRCA1 expression under the median (1.97). The median survival was 5.4 months for high expressers and 11.7 for low expression patients and the PFS 1.5 and 2.3 months respectively. The percentage of patients with PFS6 was 41.67% for those having low expression of BRCA1 and 11.54% in those with high expression. This difference is statistically significant [p=0.011]. The median survival at 12 months was 42.23% vs 35.71% respectively [p=0.632]

2. Correlation of ERCC1 mRNA Expression Levels and ET-743 Treatment Outcome.

The association between the expression levels of ERCC1 mRNA with the clinical outcome of the patients is shown in Table 2B

TABLE 2B
Association of mRNA expression of ERCC1
and patients clinical outcome.

ERCC1 mRNA

| | <5.86 | >=5.86 | Total |
|---|---|---|---|
| resp | | | |
| PR + MR | 2 | 4 | 6 |
| | 11% | 21% | |
| SD | 5 | 5 | 10 |
| PD | 12 | 10 | 22 |
| Total | 19 | 19 | 38 |
| | 50% | 50% | |

Frequency Missing = 15

| | | | |
|---|---|---|---|
| PFS | | | |
| <6 | 16 | 13 | 29 |
| >=6 | 3 | 6 | |
| | 16% | 32% | |
| Total | 19 | 19 | 38 |
| | 50% | 50% | |

Frequency Missing = 15

The amount of ERCC1 mRNA relative to the β-actin (internal control) in the 38 samples analysed ranged from 1.01 to 22.9, a 21-fold difference from the minimum to the maximum value found. The median expression value was 5.86.

Table 2B shows that 6 out of 9 (66%) patients reaching the PFS6 endpoint had ERCC1 expression values above the median value (5.86) of the sample cohort. Similarly, 4 of 6 (67%) patients having objective response (PR+MR) have expression values of ERCC1 above the median value. Regarding the distribution of objective responses across the expression of ERCC1, 6 out of 19 (32%) of patients expressing high ERCC1 reach PFS6 vs 3 of 19 (16%) of the low expression of ERCC1. Similarly, 4 out of 19 (21%) high expressers of ERCC1 had objective response (PR+MR) compared to 2 in 19 (11%) of low expression patients.

The results provided herein indicate that high levels of expression of ERCC1 has either beneficial or at least do not decrease the response of patients to ET-743 treatment. Remarkably, this correlation is opposite to that obtained with Cisplatin in NSCLC and Doxorubicin in ovarian cancer, were an increase in ERCC1 expression, meaning a higher DNA repair efficiency, is correlated to poor outcome.

The Kaplan-Meier plots of FIG. 2B show slight increase in the median PFS (1.4 vs 2.2 months for low and high expression of ERCC1 respectively [p=0.6315]) and PFS6 (21.05% vs 31.58%, [0.458]). The median survival was 19.4 months for high expressers and 16.8 for low expression patients [p=0.7682].

3. Correlation of XPD mRNA Expression Levels and ET-743 Treatment Outcome.

The association between the expression levels of XPD mRNA with the clinical outcome of the patients is shown in Table 2C

TABLE 2C
Association of mRNA expression of
XPD and patients clinical outcome.

XPD mRNA

| | <1.99 | >=1.99 | Total |
|---|---|---|---|
| PFS | | | |
| <6 | 14 | 15 | 29 |
| >=6 | 3 | 5 | 8 |
| | 18% | 25% | |
| Total | 17 | 20 | 37 |
| | 46% | 54% | |

Frequency Missing = 16

| | | | |
|---|---|---|---|
| Response | | | |
| PR + MR | 2 | 3 | 5 |
| | 12% | 15% | |
| SD | 4 | 6 | 10 |
| PD | 11 | 11 | 22 |
| Total | 17 | 20 | 37 |
| | 46% | 54% | |

Frequency Missing = 16

The amount of XPD mRNA relative to the β-actin (internal control) in the 37 samples analysed ranged from 0.8 to 13.38, a 17-fold difference from the minimum to the maximum value found. The median expression value was 1.99.

Table 2C shows that 5 out of 8 (63%) patients reaching the PFS6 endpoint had XPD expression values above the median value (1.99) of the sample cohort. Similarly, 3 of 5 (60%) patients having objective response (PR+MR) have expression values of XPD above the median value. Regarding the distribution of objective responses across the expression of XPD, 5 out of 20 (25%) of patients expressing high XPD reach PFS6 vs 3 of 17 (18%) of the low expression of XPD. Similarly, 3 out of 20 (15%) high expressers of XPD had objective response (PR+MR) compared to 2 in 17 (12%) of low expression patients.

The results provided herein indicate that high levels of expression of XPD has either beneficial or at least do not decrease the response of patients to ET-743 treatment. Remarkably, this correlation is opposite to that obtained with Cisplatin in NSCLC and Doxorubicin in ovarian cancer, were an increase in XPD expression, meaning a higher DNA repair efficiency, is correlated to poor outcome to treatment with the drug.

The Kaplan-Meier plots of FIG. 2C show slight increase in the median PFS (1.2 vs 2.0 months for low and high expression of ERCC1 respectively [p=0.5681]) and PFS6 (17.65% vs 30%, [0.3708]). The median survival was 16.8 months for low expressers and 19.4 for high expression patients.

In conclusion, the marker gene having a greater correlation to the clinical outcome is BRCA1. In fact, subdivision of the full cohort o patients in two equal subpopulations according to the BRCA1 expression produces a significant increase of the efficiency of ET-743 in the target subpopulation from 16% to 21% for objective response (PR+MR) and 24% to 38% for progression free survival higher than 6 months.

ERCC1 and XPD expression levels do not impact the clinical outcome of the ET-743 therapy, indicating that ET-743 would be equally active in those patients with poor response to Cisplatin or Doxorubicin due to the high expression levels of ERCC1 and XPD.

1. Use of ET-743 in the manufacture of a medicament for the treatment of cancer in patients having low levels of BRCA1.

2. Use of ET-743 according to claim 1, wherein the level of BRCA1 in patients is <3.

3. Use of ET-743 according to claim 2, wherein the level of BRCA1 in patients is <2.

4. Use of ET-743 according to any of the previous claims, wherein the cancer to be treated is selected from sarcoma, leiomyosarcoma, liposarcoma, osteosarcoma, ovarian cancer, breast cancer, melanoma, colorectal cancer, mesothelioma, renal cancer, endometrial cancer and lung cancer.

5. Use according to claim 4, wherein the cancer to be treated is selected from sarcoma, preferably leiomyosarcoma, liposarcoma or osteosarcoma.

6. Use of BRCA1 as a marker for the selection of cancer patients to be efficaciously treated with ET-743.

7. A method of treating cancer in a patient, comprising: assaying a biological sample from the individual for BRCA1 expression level, determining said expression level and treating the patient with ET-743 if his expression level is low.

8. The method according to claim 7 wherein the cancer to be treated is selected from sarcoma, leiomyosarcoma, liposarcoma, osteosarcoma, ovarian cancer, breast cancer, melanoma, colorectal cancer, mesothelioma, renal cancer, endometrial cancer and lung cancer.

9. The method according to claim 8 wherein the cancer to be treated is selected from sarcoma, preferably leiomyosarcoma, liposarcoma or osteosarcoma.

10. A method according to claims 7-9 wherein the biological sample is a tumour biopsy.

11. A screening method for selecting a patient suffering from cancer for a treatment with Ecteinascidin 743, comprising the steps:
a) isolating mRNA from a tissue sample of the patient;
b) determining a gene expression level of BRCA1 in the sample;
c) comparing the BRCA1 gene expression levels in the sample with a predetermined threshold level for the BRCA1 gene expression;
and classifying the patient in one of 3 groups defined as “low”, “normal” or high” according to the results of the comparison of the BRCA1 gene expression level with the predetermined threshold level.
